(19) United States
(12) Patent Application Publication
(10) Pub. No.: US 2006/0018855 A1
(43) Pub. Date: Jan. 26, 2006

(54) Cosmetic method and composition

(75) Inventors: Andrea Paula Batista, Seacroft (GB); Michael John Inger, Bebington (GB); Adrian Simon Pesa, Tortuguitas (AR); Axel Volker, Seacroft (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a UNILEVER

(21) Appl. No.: 11/184,656

(22) Filed: Jul. 19, 2005

(30) Foreign Application Priority Data
Jul. 20, 2004 (GB) 0416252.5

(51) Int. Cl. A61K 8/19; A61K 36/18

(52) U.S. Cl. 424/65; 424/776

(57) ABSTRACT

An aqueous antiperspirant or deodorant oil in water emulsion suitable for being dispensed through a roll-on exhibiting superior consumer attributes and comprising a continuous aqueous phase containing an antiperspirant or deodorant active, an ethoxylated ether emulsifier or mixture having an average HLB of from 7 to 9 and a dispersed oil phase comprising a plant oil such as a fatty acid triglyceride and a particulate silica and especially a hydrophobic fumed silica.

The present invention relates to a cosmetic method and composition, and in particular to antiperspirant or deodorant formulations and to cosmetic methods of controlling body odour or perspiration from localised areas of the body, such as in the underarm.

Sweating is a natural bodily function of humans which results in the formation of visible wet patches on human skin or in clothing which comes into contact with wet or damp skin. The appearance of such wet patches is considered undesirable in a number of societies in many circumstances and accordingly an industry has become established to produce materials, commonly called antiperspirants, which control sweating, especially in those skin regions where the density of sweat glands is particularly high, such as in the underarm.

Moreover, even though freshly secreted sweat tends to have little odour, the resident skin population of micro-organisms and particularly certain stains of bacteria act on the secretions to transform at least a fraction of them into malodorous compounds, and especially in occluded sites in the body such as in the underarm. Accordingly, a parallel industry has become established in which compositions, commonly called deodorants, are contacted with the skin so as to mask such malodours or to prevent or retard their formation. By virtue of the suppression of sweating, antiperspirants usually additionally provide localised deodorancy.

Antiperspirant or deodorant dispensers commonly fall into one or other of two categories, namely contact or non-contact dispensers. Choice between those two categories tends to be a matter of personal preference since safe and effective formulations are available in either category. Within the category of contact dispensers, there is a range of alternative classes of dispenser depending on the physical characteristics of the composition, including sticks in which the composition forms a solid mass which can retain its integrity unsupported by a container, and soft solids or creams which are either very viscous or thixotropic which can be retained within a dispensing container but dispensed therefrom through apertures or slits by mild pressure. A third class of dispensers and compositions comprises liquids with a relatively low viscosity that commonly are dispensed via a roller that sits loosely enough within a housing forming an outlet from the dispenser to allow it to rotate when rolled across the skin, often called roll-ons.

The instant invention relates to compositions that can be dispensed by roll-ons. Many roll-on deodorant or antiperspirant compositions have been based on a low molecular weight alcohol such as ethanol as the principal carrier and/or active ingredient in the composition, by which herein we mean the ingredient providing the largest single weight fraction. It has bactericidal properties and is a good solvent to common antiperspirant actives. However, it has a number of characteristics which are disliked by or repugnant to a significant fraction of prospective deodorant or antiperspirant users. These characteristics include stinging, especially if the skin is broken or abraded, and a significant cooling effect. For that reason, since the perception by consumers of cosmetic compositions can influence to a considerable extent whether or not they purchase the same brand again, it is desirable to devise compositions for consumers who wish to avoid ethanol-based compositions. Some alternative formulations are aqueous emulsions but they can suffer from one or more unfavourable characteristics such as at least a perception of drying too slowly and/or greasiness.

WO03/041674 discloses a roll-on formulation of the water-in-oil type. Water-in oil formulations are known to the skilled man to present different problems from oil in water cosmetic formulations; for example, they act differently because the external phase is different. Not surprisingly, therefore, it is silent about the problems of drying indicated above.

U.S. Pat. No. 0,108,580 is directed to a process for the preparation of granulates of pyrogenically-produced silicon dioxide and to the use of such granulates in cosmetic compositions, exemplifying creams and powders. It is silent about the problems of drying oil-in-water emulsions.

U.S. Pat. No. 5,849,276 discloses the use of silica as a nucleating agent in anhydrous antiperspirant compositions. Silica has previously been incorporated into anhydrous antiperspirant stick formulations (JP55004355) and anhydrous deodorant compositions (JP01143820). Each of these three specifications is silent about the problems of drying emulsions indicated above.

It is an objective to obtain a composition that is suitable for employment in a roll-on dispenser and which at least ameliorates one or more unfavourable or disliked characteristics of such compositions, such as one or more mentioned hereinbefore.

According to the present invention there is provided a cosmetic composition in accordance with claim 1 herein. By the incorporation of particulate silica into such an emulsion, it is possible to improve or ameliorate one or more unfavourable characteristics of corresponding silica-free emulsions.

According to another aspect of the present invention there is provided a method for improving the drying characteristics of an oil-in-water antiperspirant or deodorant emulsion in accordance with the method of claim 31.

The instant invention employs an effective concentration of an antiperspirant or deodorant active, which is say that is sufficient to reduce or control sweating or reduce or eliminate body malodour. In many desirable embodiments, the composition contains at least 1% antiperspirant active, and preferably a least 5% and often is at least 10%. Commonly, the concentration of the antiperspirant active is not higher than 30%, and in many practical embodiments is not higher than 25.5%, %s herein being by weight based on the composition unless otherwise stated. A preferred concentration range for the antiperspirant active is from 10 to 20%.

The antiperspirant active is conveniently an astringent aluminium and/or zirconium salt, including astringent inorganic salts, astringent salts with organic anions and complexes of such salts. Preferred astringent salts include aluminium, zirconium and aluminium/zirconium halides and halohydrate salts, such as especially chlorohydrates. Activated chlorohydrates can be incorporated, if desired. Some literature employs alternative terminology for chlorohydrates, such as basic aluminium chloride, and aluminium chlorhydrex.

Aluminium halohydrates are usually defined by the general formula Al₂(OH) ₓQ_{y}.wH₂0 in which Q represents respectively chlorine, bromine or iodine, (and especially chlorine to form a chlorohydrate) x is variable from 2 to 5 and x+y=6 while wH₂O represents a variable amount of hydration.

Zirconium actives can usually be represented by the empirical general formula: ZrO(OH) _{2n-nz}B_{z}.wH₂0 in which z is a variable in the range of from 0.9 to 2.0 so that the value 2n−nz is zero or positive, n is the valency of B, and B is selected from the group consisting of chlorine (to form a chlorohydrate), other halide, sulphamate, sulphate and mixtures thereof. Possible hydration to a variable extent is represented by wH₂0. Preferably, B represents chlorine and the variable z lies in the range from 1.5 to 1.87. In practice, such zirconium salts are usually not employed by themselves, but as a component of a combined aluminium and zirconium-based antiperspirant.

The above aluminium and zirconium salts may have coordinated and/or bound water in various quantities and/or may be present as polymeric species, mixtures or complexes.

In particular, zirconium hydroxy salts often represent a range of salts having various amounts of the hydroxy group. Zirconium aluminium chlorohydrate may be particularly preferred.

Antiperspirant complexes based on the above-mentioned astringent aluminium and/or zirconium salts can be employed. The complex often employs a compound with a carboxylate group, and advantageously this is an amino acid. Examples of suitable amino acids include dl-tryptophan, dl-β-phenylalanine, dl-valine, dl-methionine and β-alanine, and preferably glycine which has the formula CH₂ (NH₂) COOH.

In some compositions, it is highly desirable to employ complexes of a combination of aluminium chlorohydrates and zirconium chlorohydrates together with amino acids such as glycine, which are disclosed in U.S. Pat. No. 3,792,068 (Luedders et al). Certain of those Al/Zr complexes are commonly called ZAG in the literature. ZAG actives generally contain aluminium, zirconium and chloride with an Al/Zr ratio in a range from 2 to 10, especially 2 to 6, an Al/Cl ratio from 2.1 to 0.9 and a variable amount of glycine. Actives of this preferred type are available from Westwood, from Summit and from Reheis.

The invention compositions can comprise, if desired, a deodorant active other than an antiperspirant active described hereinbefore. Such an alternative deodorant active can be selected conveniently from any deodorant active known in the cosmetic art such as antimicrobial actives such as polyhexamethylene biguanides, e.g. those available under the trade name Cosmocil™ or chlorinated aromatics, eg triclosan available under the trade name Irgasan™, non-microbiocidal deodorant actives such as triethylcitrate, bactericides and bacteriostatis. Yet other deodorant actives can include bactericidal zinc salts such as zinc ricinoleate. The concentration of such alternative deodorant active is desirably from 0.01 to 5% and in many instances is from 0.1 to 1% by weight of the composition.

In many highly desirable invention compositions, an antiperspirant active is present, either without or supplemented by the alternative deodorant active.

An essential constituent of compositions of the present invention is a non-ionic emulsifier or mixture of emulsifiers forming an emulsifier system. Such an emulsifier system conveniently has a mean HLB value in the region of from about 5 to about 12 and particularly from 6 to about 10. An especially desired mean HLB value is from 7 m to 9. Such a mean HLB value can be provided by selecting an emulsifier having such an HLB value, or more preferably by employing a combination of at least two emulsifiers, a first (lower) HLB emulsifier having an HLB value in the range of from 2 to 6.5, such as in particular from 4 to 6 and a second (higher) HLB emulsifier having an HLB value in the range of from about 6.5 to 18 and especially from about 12 to about 18. When a combination of emulsifiers is employed, the average HLB value can be obtained by a weight average of the HLB values of the constituent emulsifiers.

An especially desirable range of emulsifiers comprise a hydrophilic moiety provided by a polyalkylene oxide (polyglycol), and a hydrophobic moiety provided by an aliphatic hydrocarbon, preferably containing at least 10 carbons and commonly linear. The hydrophobic and hydrophilic moieties can be linked via an ester or ether linkage, possibly via an intermediate polyol such as glycerol.

Preferably the hydrophobic aliphatic substituent contains at least 12 carbons, and is derivable from lauryl, palmityl, cetyl, stearyl, olearyl and behenyl alcohol, and especially cetyl, stearyl or a mixture of cetyl and stearyl alcohols or from the corresponding carboxylic acids. It is particularly convenient to employ an emulsifier comprising a polyalkylene oxide ether.

The polyalkylene oxide is often selected from polyethylene oxide and polypropylene oxide or a copolymer of ethylene oxide and comprises a polyethylene oxide. The number of alkylene oxide and especially of ethoxylate units within suitable emulsifiers is often selected within the range of from 2 to 100. Emulsifiers with a mean number of ethoxylate units in the region of 2 can provide a lower HLB value of below 6.5 and those having at least 4 such units a higher HLB value of above 6.5 and especially those containing at least 10 ethoxylate units. A preferred combination comprises a mixture of an ethoxylate containing 2 units and one containing from 10 to 40 units. Particularly conveniently, the combination of emulsifiers comprises steareth-2 and a selection from steareth-15 to steareth-30.

It is desirable to employ a mixture of ethoxylated alcohol emulsifiers in a weight ratio of emulsifier having a lower HLB value of <6.5 to emulsifier having a higher HLB value of >8 of from 1.5:1 to 6:1 and particularly from 2:1 to 5:1.

The total proportion of emulsifiers in the composition is usually at least 1.5% and particularly at least 2% by weight. Commonly the emulsifiers are not present at above 6%, often not more than 5% by weight and in many preferred embodiments up to 4% by weight. An especially desirable concentration range for the emulsifiers is from 2.5 to 4% by weight.

An other essential constituent of the present invention compositions is an oil. The oil is advantageously a plant oil and particularly is a triglyceride oil. Such oils are often obtainable by extraction from the plant's seeds. Suitable plant oils include sunflower seed oil, maize corn oil, evening primrose oil, coriander seed oil, safflower oil, olive oil, rape seed oil, castor oil and borage seed oil. It is particularly desirable to employ an oil which comprises mono or polyunsaturated long chain aliphatic carboxylate substituents, such as notably C18 carboxylates containing 1, 2 or 3 degrees of unsaturation, 2 or more or which may be conjugated. Other suitable oils which come into consideration include jojoba oil. The proportion of oil in the composition (excluding any contribution from water-insoluble constituents of fragrance oils which may be present) is often at least 1% and commonly a least 1.5% by weight. In many instances the proportion of oil is not more than 10% by weight and notably is not more than 5% by weight.

A further essential constituent of the composition comprises a particulate silica such as an amorphous silica, eg a fumed silica. It is particularly desirable to employ such a fumed (sometimes called pyrogenic) silica which has been hydrophobically treated. Such materials are commercially available under the name hydrophobic silica. Hydrophobic silicas are obtained by chemically bonding a hydrophbic substituent such as especially a siloxane group onto the surface of the silica, possibly following an intermediate treatment in which the surface of the silica has been rendered hydrophilic. Suitable reactants to generate a hydrophobic substituent include halosilanes and in particular chlorosilanes and methylated silazanes such as hexamethyldisilazane. It is particularly desirable to employ a silica that is capable of thickening an oil such as a plant oil.

Desirably, the silica, such as the fumed silica, and especially the hydrophobic silica has a BET specific surface area of at least 100 m²/g and particularly from 150 to 400 m²/g. The silica comprises very fine particles, fumed silica commonly having a diameter for individual particles of below 40 nm and in many instances at least 99% by weight of below 40 nm. In fumed silica as supplied, some aggregation can occur so that in many embodiments, the supplied silica has an average particle size (diameter) of less than or equal to 1000 nm, preferably less than or equal to 500 nm, i.e. the diameter of the silica particle of average weight. In at least some desirable embodiments, at least 99% by weight of the silica particles, as supplied, are in the range of 10 to 500 nm.

The weight proportion of silica in the formulation is often selected taking into account the desired viscosity of the eventual formulation, together with other attributes such as its effect on the speed of drying of the formulation, its perceived greasiness and/or its perceived stickiness. The weight concentration of silica in the composition is desirably at least 0.2%, often at least 0.3% and in many desirable embodiments is at least 0.5% by weight. Its concentration is commonly not greater than 2%, often not greater than 1.5% and in a number of very desirable formulations is not higher than 1.0%. A preferred weight range of silica concentrations is from 0.6 to 0.8%.

The water content of the composition is commonly selected in the range of from 65 to 93% by weight and often from 70 or 75 to 85% by weight.

The weight ratio of silica to water in the invention emulsions is commonly selected in the range of at least 1:400 up to 1:40, often at least 1:275 and in many instances preferably at least 1:200. It is often convenient to employ a weight ratio of up to 1:75.

In addition to the foregoing essential constituents it is preferable to include a fragrance, for example in a proportion of from 0.1 to 3% by weight, and particularly from 0.3 to 2% by weight.

In a number of highly desirable embodiments, the invention compositions comprise, by weight:

from 70 to 85% of water;

from 10 to 20% of an antiperspirant active, such as actives described hereinbefore;

from 2.5 to 4.0% of an ethoxylated ether emulsifier or mixture of emulsifiers, preferably having an HLB value of from 7 to 9;

from 1.5 to 4% by weight of a plant oil, such as an unsaturated fatty acid triglyceride;

from 0.5 to 1.0% of a hydrophobic fumed silica and

from 0.3 to 2% of a fragrance.

By the selection of the proportions of the above identified constituents within the foregoing disclosed ranges of proportions, it is possible to obtain emulsions having a viscosity which fall within a preferred range of from 1000 to 7000 mPa·s and particularly within 2500 to 5500 mPa·s. Viscosities herein are measured in a Brookfield RVT viscometer equipped with a stirrer TA and Hellipath, rotating at 20 rpm at 25° C. unless otherwise stated. Such emulsions demonstrate a particularly desirable combination of product attributes such as improved speed of drying, superior greasiness and avoidance of excessive stickiness on application.

According to a second aspect of the instant invention there is provided a process for making an emulsion in accordance with claim 23 herein. The steps of mixing and shearing can be carried out using the relevant conventional equipment.

Preferably, the emulsion is made by first preparing separate aqueous and oil mixtures which are brought together before shearing. The aqueous phase commonly contains the antiperspirant active. Where a mixed emulsifier system is employed, it is desirable to incorporate any emulsifier having a low HLB value, particularly of <6.5 into the oil phase and an emulsifier having a high HLB value, particularly of >6.5 into the aqueous phase. The temperature of the respective phases can be raised, where necessary, to accelerate dissolution of the emulsifier, for example to above 50° C.

It is highly desirable to incorporate the silica and especially hydrophobic silica, with the aqueous phase. It is preferable to incorporate any fragrance last of all and shortly before the entire mixture is sheared, especially when either or both phases have been heated so as to accelerate emulsifier dissolution.

The invention formulations are very suitable for dispensing via a roll-on dispenser, for example any dispenser such as described in EP1175165 or an invert dispenser such as described in U.S. Pat. No. 6,511,243.

The instant invention provides an antiperspirant or deodorant product which comprises a dispenser comprising a reservoir containing an oil in water fluid emulsion composition according to the first aspect said reservoir having an outlet for the composition defining

a housing for a roller

a roller retained by the housing and partially proud of the outlet

a means for releasably sealing the outlet and

a cap for the outlet having an exterior profile that permits the dispenser to stand stably in an invert orientation.

Upright herein indicates that when the dispenser is standing on a surface, the reservoir is below the housing for the roller, which in turn is below the cap, whereas Invert indicates the opposite.

The sealing means conveniently comprises a screw threaded connection between the interior of the cap and the exterior of the housing so as to urge the roller into fluid-tight contact with the housing. The roller most conveniently comprises a sphere and the housing most conveniently has a radiuses inner surface dimensioned so as to allow a small gap between the roller and the housing when the outlet is not sealed. When the roller is urged inwardly towards the housing, for example by suitably rotating the cap about the exterior of the housing, the screw threads engage, the cap urges the roller inwardly and the gap is sealed.

The invention formulation are particularly suited to being dispensed from a dispenser comprising one or more perturbators to regulate fluid flow and particularly gaseous flow within the housing for the ball and/or a means to control the depth of liquid adhering to the ball.

In some preferred embodiments of the present invention, the invention formulations are dispensed from a roll-on dispenser for a liquid comprising a bottle and a removable cap, the bottle having an interior and a first end which defines a housing for a rotatable spherical ball, said housing having a chamber within which the ball can rotate having an inward end in fluid communication with the interior of the bottle and comprising a lateral sealing ring dimensioned to prevent the ball being urged into the interior of the bottle when subject to inward axial force, an opposed outward end dimensioned to retain the ball and a side-wall having an interior surface extending between the outward end and the inward end the ball having a segment projecting outside the housing, the cap having means to urge the ball axially towards the sealing ring, in which at least one fluid flow perturbator is located on the interior surface of the chamber in or outward of the sealing ring.

Herein the term “inward” when employed axially, for example in “inward end” in respect of a housing intended for mounting on or an integral mounding with a bottle reservoir refer respectively to the end adjacent to the interior of the bottle, and outward is that remote from the interior. Axial relates to an axis extending centrally through the inward and outward ends of the housing.

Herein, the terms upward, downward, above and below when employed in respect of the dispenser and its constituent parts refer to when the dispenser is in an upright orientation, which is to say the cap is above the bottle.

In such preferred embodiments, the fluid flow within the housing is modified by incorporating at least one perturbator within the housing chamber, including, in particular, modifications to the interior surface of the housing for the ball which result in localised disruption of or modification the flow of fluids across that surface.

The interior surface of the housing chamber can be modified in a number of different ways to perturb fluid flow over the surface. This can comprise the introduction of baffles, preferably baffles on the interior face which can possibly introduce turbulence into the fluid flow. Conveniently, the baffles can comprise one or more continuous or discontinuous lateral beads intermediate between the outward end of the housing and the lateral sealing ring. If desired, the beads can also be castellated or have an axial component, as for example the beads being arranged in chevrons or an array of short sections distributed symmetrically or randomly across the interior face of the housing. The beads commonly have a depth of from about 50 to 500 μm, so as to achieve fluid perturbation but not obstruct the outward of flow of liquid from the dispenser excessively. Where lateral beads are employed, such as two beads, their axial spacing from each other is often selected in the range of from 2 to 8 mm such as from 2.5 to 4.5 mm. The bead or closest bead is often in the range of from 2 to 8 mm axially outward from the sealing ring and particularly from 2.5 to 4.5 mm. The or closest bead is often from 5 to 10 mm axially inward of the outward end of the housing.

A further and especially desirable way in which the ball housing surface can be modified to cause fluid perturbation in the chamber comprises notching the lateral sealing ring with a multiplicity of axially extending notches at its upward edge i.e. the edge pointing towards the outward end of the housing. The notches are preferably made all around the sealing ring, and especially they are approximately equidistant from their neighbours. The number of notches is at the discretion of the manufacturer, but preferably selected to provide a spacing in the range of from 2.5 to 7.5 mm and particularly in the range of from 3 to 5 mm for hand-held cosmetic dispensers employing a roll-ball of from 25 to 35 mm diameter such as is particularly suitable for dosing cosmetic products such as deodorants and antiperspirants. For a sealing ring having a circumference of from 90 to 110 mm, this indicates the number of notches conveniently being in the range of from 15 to 36, such as particularly from 20 to 30.

The notches are dimensioned and profiled, in practice, so as to encourage fluid turbulence. The width of each notch is often from about 250 or 500 to 2000 μm, such as from 750 to 1500 μm. The axial depth of each notch is often at least 500 μm and commonly not greater than 2500 μm, and in a number of preferred embodiments is from 1200 to 1600 μm. The notches advantageously have a sharp outward edge, such as from about 85 to 110°, and preferably square edged. The notches themselves are commonly rectangular in cross section, possibly having tapering sides.

Advantageously, the notches on the sealing ring are in fluid connection with an annular, preferably v- or u-shaped groove formed in the side-wall of the housing beside the upward-facing edge of the sealing ring and. This lateral v- or u-shaped groove conveniently has a width (at its upward end, i.e. its mouth) and axial depth which are each desirably selected in the range of from 500 to 2500. Its depth is advantageously similar to the depth of the notches in the sealing ring, and preferably is the same.

Advantageously, the housing employs both the baffles and the notched sealing ring.

The instant invention may in at least some embodiments be considered to comprising the provision of a housing for a roll-on which comprises one or more baffles such as a bead or beads as described herein and/or, especially, the notching of the outward facing edge of a sealing ring on the interior face of the housing as described herein, particularly when it is in communication with an annular groove, irrespective of the reason why they smooth variations in the dose of a cosmetic liquid applied by the dispenser.

The housing can additionally have, mounted therein, a spider positioned between the sealing ring and the interior of the bottle. Such a spider can comprise a plurality of spokes, optionally with an intermediate ring linking two or more of the spokes. The spokes can be fixed, that is to say have both ends secured, for example one end being mounted on the interior face of the housing and the other end fixed to a hub, or can be free, which is to say has one end fixed, for example being mounted to the housing wall or to the aforementioned hub. The spider can have a flat, concave or convex profile facing the ball. However, it is particularly desirable to employ a spider having a convex profile and especially a spider in accordance with the disclosure in a co-pending application of even date entitled “Improvements in a Cosmetic Dispenser”, any supplementary description and accompanying drawings compared with the present text and drawings being incorporated herein by reference. Use of such a particularly desirable spider, that can wipe excess liquid off the ball to leave a film of pre-determined depth, can contribute to the reduction of fluctuations between successive topical applications of the cosmetic formulation.

A spider herein comprises a plurality of spokes which can as a minimum be two, provided that together they subtend a wide arc of the housing, such as preferably at least 120 through to 240 degrees and preferably are at or close to 180 degrees apart. Preferably, the number of spokes is at least 3, and in some instances, the number of spokes is at least 4 The number of spokes is normally no greater than 12, in so as not to constrict the passage of liquid between the spokes unduly, and in several preferred embodiments is not more than 9. A convenient number is 3, 4, 5 or 6 spokes, and especially 6 spokes. Although the spokes can be arranged asymmetrically around the interior of the housing side-wall, it is preferable to employ a symmetrical arrangement, for example point or mirror symmetry.

The spider can be mounted on the interior of the housing side-wall one or more mounting points. When a single mounting point is employed, the spoke leading away from the mounting point terminates at its opposed end in a hub from which radiates at least one further spoke, and preferably from 2 to 5 further spokes. Preferably the spider is mounted on the side-wall at two or more mounting points that themselves are preferably symmetrically arranged around the interior side-wall and conveniently by 3 or 4 mounting points. The mounting points are most desirably equidistant around the housing side-wall and lateral relative to each other, i.e. all at the same axial distance below the widest diameter of the housing.

In many embodiments, the spider comprises a hub from which spokes radiate towards the housing side-wall. If desired, all the spokes can extend between the side-wall and the hub, and for convenience herein these can be called fixed spokes. However, some of the spokes that radiate from the hub and spokes that radiate towards the centre of the housing from the side-wall can have a free end, by which free end is meant that it is not secured to respectively the side-wall or the hub, and for convenience herein these can be called free spokes. It is preferred to employ a mixture of free and fixed spokes, for example in a ratio of from 1:2 to 2:1, and conveniently at 1:1. The free spokes tend to be more flexible whereas the fixed spokes tend to be more rigid and assist in the production of the combined housing and spider, for example in injection moulding. It is especially desirable for fixed and free spokes to arranged symmetrically, such as 1 or 2 free spokes interposed between adjacent fixed spokes. By adopting a symmetrical arrangement, the ball can be centred more easily, thereby ensuring best that the spokes control the depth of liquid film more evenly. One especially desirable arrangement comprises an even number of spokes in total being 4, 6 or 8 having alternate fixed and free spokes symmetrically arranged around the side-wall.

The spider desirably has a means for providing localised contact with the ball and especially when it has a concave upward, ball-facing surface. Preferably, the spider, is resilient and flexible, at least in an axial direction, or constituent spokes thereof, and mounted so as to biass the ball upwardly, thereby keeping the spider in localised contact with the ball, when the cap is removed. The downward force exerted on the ball maintains contact between ball and spider when the cap is fitted, flexing downwardly the spider, or at least the spokes carrying the boss or pimple.

The localised contact means desirably comprises a boss or pimple standing proud of the surface of the spider, specifically proud of the surface of the spokes facing the ball. The boss or pimple is desirably of round or rounded lateral cross section. The boss or pimple advantageously has a bevelled or rounded chamfer to its contact edge with the ball, thereby to minimise frictional contact with the ball. The boss or pimple advantageously is hemispherical or a cylinder terminating in a hemisphere. The orthogonal height of the pimple, which controls the depth of the liquid film adhering to the ball, is often, for a hand-held cosmetic dispenser, selected in the range of from 300 to 2000 μm and in many instances from 350 to 750 μm.

The pimple or boss often has a diameter of from 300 to 2500 μm and particularly from 350 to 1000 μm, often tapering or rounded to a point (such as below 25 μm diameter) for contact with the ball. The boss or pimple can be employed on free or fixed spokes and on the hub. Advantageously, at least one pimple or boss per spoke is located at a point that is remote from the point of attachment of the spoke, he free to the hub and the fixed to the housing side-wall as the case may be. Particularly desirably each free spoke has a pimple. Most desirably, the pimples present a symmetrical pattern. If desired any spoke can be provided with a plurality of pimples, such as 2 or 3. Preferably when the housing comprises an annular sealing ring between the ball and the spider mounting point(s), any pimple on a spoke mounted on the wall is equidistant between the wall and centre of the housing or closer to the centre.

Alternatively, for a free spoke, the means for spacing the spoke surface from the ball surface can comprise the tip of the spoke being bent upwardly towards the ball, desirably to provide a similar spacing to that provided by a pimple. In the vicinity of the hub, the localised contact can be provided by an upstanding wall that is either continuous or discontinuous and concentric with the housing side-wall.

Desirably a free spoke extends at least about 40% the radius of the interior of the housing, so as to be relatively flexible. In many embodiments the free spoke extends up to 95% of the housing interior radius and especially from 75 to 95% particularly when it extends from a hub. When it extends from the side-wall, the free spoke particularly extends from 50 to 80% of the housing interior radius.

The spokes desirably have a triangular side profile with a concave top wall preferably matching the radius of the ball with the apex of the triangle at the hub, or closest to the hub for a free spoke that is mounted on the side-wall. Such a profile assists the spoke to flex whilst strengthening it adjacent to its mounting point. The spokes can desirably comprise an upstanding wall, often tapered and a base plate, providing a T-shaped cross section. Transversely, the spoke top can be flat or concave, such as with a radius of curvature similar to that of the ball. Advantageously, the spoke sides can be approximately orthogonal to the ball tangent.

Preferably, the concavity of the ball-facing top surface of the spokes has a similar radius to that of the ball so as to enable the film depth to be substantially the same along the length of the spokes. The mounting point of the spokes on the side-wall is such that the ball is radially spaced from the mounting point at the same height as the pimple or like gap-forming means.

The resilient spider often provides a contact force (acting upwardly on the ball) of from 0.01 to 0.1 kg-f and especially in the region of 0.04 to 0.06 kg-f, a force sufficient to maintain contact, but not so great as to render operation of the dispenser difficult.

The invention is directed in particular to the use of balls having a diameter of from about 20 to about 40 mm and especially from about 25 to about 36 mm. Representative ball diameters are 25, 29, 32 or 35.5 mm, or thereabouts.

The housing and spider are preferably injection moulded together in a unitary mould, employing a thermoplastic polymer such as polyethylene or polypropylene.

In a further aspect of the present invention, there is provided a cosmetic, ie non therapeutic process for controlling perspiration or reducing body odour by dispensing a composition according to the first aspect of the present invention onto skin surface, and especially to axillary skin. This can be achieved in a conventional manner employing a roll-on dispenser as described herein.

Having described the invention in general terms, specific embodiments will now be described in more detail by way of example only.

### EXAMPLES

The Examples and comparison compositions were made by the following general method:

An aqueous phase was prepared by mixing together in a vessel a 50% aqueous solution of an astringent antiperspirant active, water and any emulsifier having a high HLB value, (>6.5) and heating the mixture until the emulsifier dissolved, typically in the region of 55 to 65° C. Any silica was incorporated into this aqueous mixture. In a second vessel, an oil phase was prepared by mixing the selected oil with any emulsifier having a low HLB value (<6.5) and heating the mixture until the emulsifier dissolved, conveniently also in the region of 55 to 65° C. The oil phase was then slowly introduced with continuous stirring into the first vessel. The resultant mixture was allowed to cool to below 40° C. and any fragrance was added. The resultant mixture was then passed through a high shear mixer to form an emulsion and charged into roll-on dispensers as described in EP 1175165. Example 4 was made by a variation of the general method in which the hydrophobic silica was added in the oil phase.

The composition of products Ex 1 to 4 and Comp A are summarised in Table 1 below.

TABLE 1

| | | | | | Comp | |
|---|---|---|---|---|---|---|
| | Ex 1 | Ex 2 | Ex 3 | Ex 4 | A | Comp B |
| Ingredient | % by weight | | | | | |
| Aluminum Chlorohydrate | 30.0 | 30.0 | 30.0 | 30.0 | 30.0 | 30.0 |
| (50% w/w solution)*¹ | | | | | | |
| Steareth-2*² | 2.3 | 2.3 | 2.0 | 2.0 | 2.3 | 2.6 |
| Helianthus Annuus*³ | 2.0 | 2.0 | 4.0 | 4.0 | 2.0 | 4.0 |
| Steareth-20*⁴ | 0.9 | 0.9 | 0.5 | 0.5 | 0.9 | 0.6 |
| Hydrophobic Silica*⁵ | 0.7 | 0.3 | 0.7 | | | |
| Hydrophobic Silica*⁶ | | | | 0.7 | | |
| PVM/MA copolymer | | | | | 0.31 | 0.31 |
| solution*⁷ | | | | | | |
| fragrance | 0.8 | 0.8 | 0.8 | 0.8 | 0.8 | 0.8 |

| | |
|---|---|
| Water | balance to 100% |

*¹Chlorhydro1 ™ sal-Reheis
*²Tego Alkanol S2 ™-Degussa
*³high oleic-Henry Lamotte
*⁴Brij 78 ™-Uniquema
*⁵HDK H30 ™-Wacker Chemie
*⁶Aerosil R8200 ™-Degussa
*⁷Gantrez S95 ™ sol-ISP

All the Examples and comparison formulations had a viscosity in the range of 1500 to 5500 mPa·s at 25° C.

Sensory Test

The sensory properties of Comparison A and Examples 1 and 2 were then compared in a head to head test.

In the sensory test, 15 expert and experienced panellists assessed inter alia the greasiness of the formulations by stroking the roll-on across the underarm in a standardised way to deposit a dose of approximately 0.3 g product, determined by weighing the dispenser before and after dose application. Products were blind coded, the order of test presentation fully randomised, and products were tested in duplicate, in order to increase data reliability. The panellists assessed greasiness on a scale of 1 to 100 on application and at minute intervals for a short while thereafter. The data was then analysed using a commercial statistical programme and the results on application and after 1 minute are summarised in Table 2 below, in which ^{SD} indicates that the improvement was statistically significant at the 95% confidence limit.

TABLE 2

| | Comp A | | Ex 1 | | Ex 2 | |
|---|---|---|---|---|---|---|
| | | | | Std | | |
| Formulation | Mean | Std dev | Mean | dev | Mean | Std dev |
| Initial (t = 0) | 29.33 | 8.38 | 27.00 | 6.45 | 25.13^{SD} | 5.64 |
| After 1 min | 28.27 | 7.56 | 23.80^{SD} | 7.49 | 23.87^{SD} | 6.63 |

Table 2 shows that the formulations incorporating the hydrophobised silica particles were less greasy than the comparison product Comp A which did not contain any silica particles, but otherwise contained the same proportions of the other active ingredients, water being considered not to be active. The improvement was at least directional on application and statistically significant after only 1 minute.

Similar sensory were carried out for Examples 3 and 4 compositions, and it was found that they again showed at least a directional improvement in greasiness, though tending to take longer to reach an improvement that was statistically significant than the formulations of Examples 1 or 2.

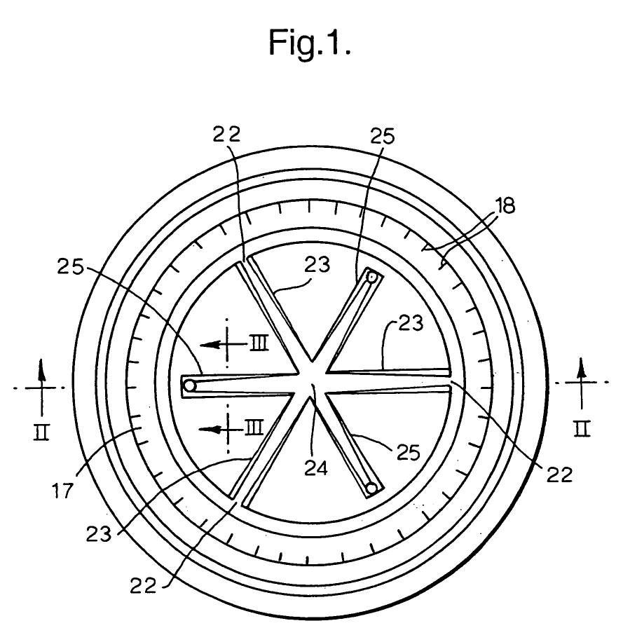

The actual and perceived speed of drying of the invention formulation of Example 1 dispensed from an applicator, as described herein with respect to FIGS. 1 to 4, was compared with a comparison formulation Comp B dispensed from a dispenser according to the example of WO 00/64302. The formulations were applied employing the same procedure as described previously herein for assessing their greasiness. Panellists determined the time of actual drying by touching the underarm and recording when the skin was dry to the touch of their fingers, and the perceived time of drying was recorded when panellists' skin sensation indicated to them that the underarm was dry, without touching it with their fingers. The recorded times were averaged and rounded to the nearest minute.

The comparison product Comp B achieved an actual speed of drying of 8 minutes and a perceived speed of drying of 9 minutes, whereas the invention product Ex 1 had an actual speed of drying of 6 minutes and a perceived speed of drying of 7 minutes. This shows that the invention product dried significantly more quickly than the comparison product, whether it was measured by touch or by skin sensation.

In the drawings for the dispenser of the invention composition employed in the drying test:

FIG. 1 is a plan view of a housing for a roll ball suitable for mounting on a cosmetic bottle, without the ball in place;

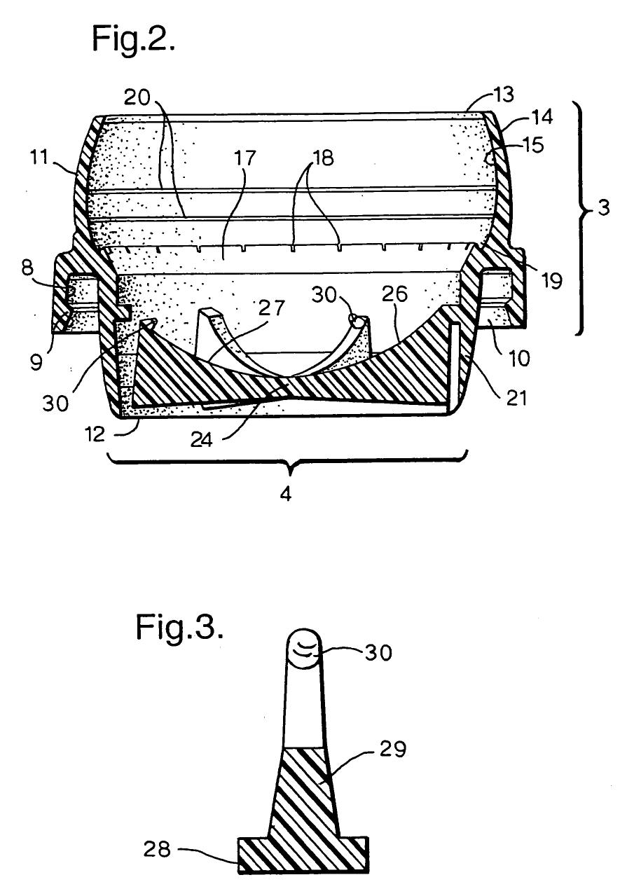

FIG. 2 is a cross section view of the housing of FIG. 1;

FIG. 3 shows a transverse cross section through a free spoke shown in FIG. 1;

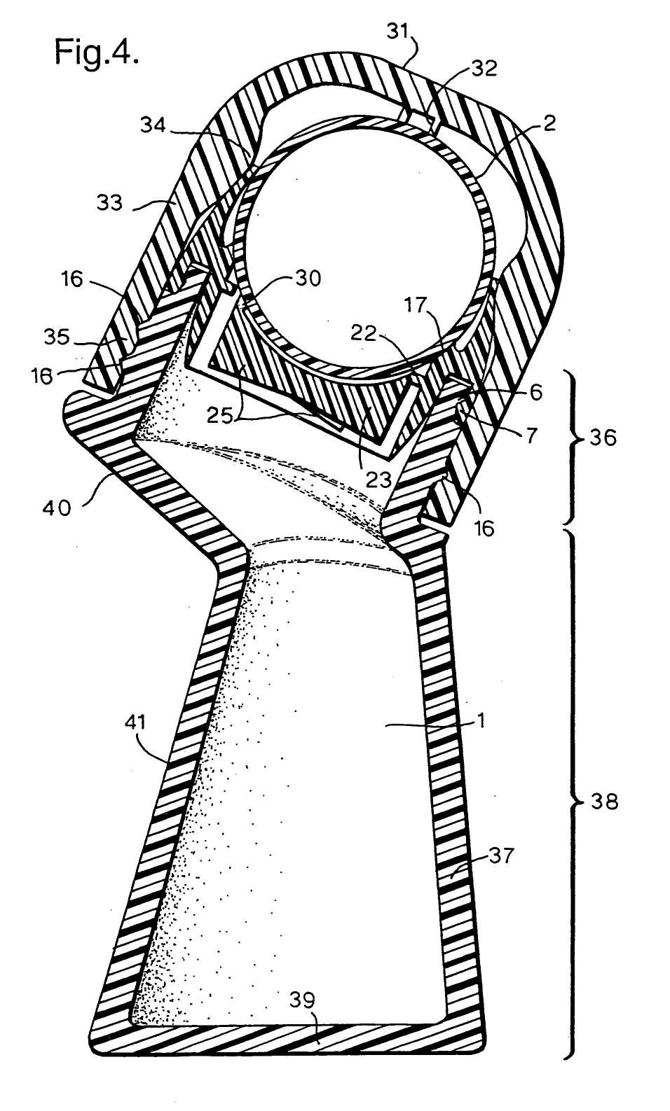

FIG. 4 is a cross section view of a dispenser showing the housing of FIGS. 1 and 2 with ball in place, mounted on a bottle and having a cap screwed tight.

As illustrated in the Figures, the roll-on dispenser comprised a bottle (1), a spherical ball (2), a housing (3) for the ball (2) integrally moulded with a spider (4) and a cap (5), each of which are moulded from a thermoplastic polymer.

The bottle (1) at its open end has an exterior annular lip (6) and annular groove (7) which snap fit with co-operating annular recess (8) and bead (9) moulding on the inward-face of an annular channel (10) formed by a bifurcated side-wall of the housing (3) dimensioned for a fluid tight fit with the bottle.

The housing (3) comprises an annular side-wall (11) of circular lateral cross section extending between an inward end (12) and an outward end (13). The side wall (11) comprises an upper wall of tapering cross section (14) adjacent to the outward end (13) which has a concave interior face (15) having two lateral shallow beads (20) and screw threads (16) on an exterior face. Shallow annular beads (20) act as baffles which perturbs air as it flows into the bottle across the interior face of the housing during use and thereby smooth the application of liquid. The housing (3) has a thickened middle wall section having an inward facing annular sealing ring (17) into the outward (upper) edge of which are cut a multiplicity of short notches (18) equidistantly spaced around the ring extending down about 30% of the axial height of the sealing ring (17), which disrupt the flow of air across the interior face of the housing. The notches (18) are of about the same depth as and in fluid communication with a lateral annular v-shaped groove (19) is defined by an inward face of the upper wall (11) and an outward face of the ring (17), which also provides a small intermediate reservoir for liquid when the dispenser is in an upright orientation. The housing side-wall is bifurcated, providing an inner annular skirt (21) extends into the bottle (1) from the middle section of the housing (3) on which a spider (4) is mounted at three equidistant points (22) around the skirt (21), which spider (4) extends across the inward end (12) of the housing.

The spider (4) comprises three fixed arms (23) extending from the housing skirt (21) to a hub (24) from which radiates three free spokes (25) that each are equidistant from the adjacent fixed spokes (23) and extend about 90% of the distance from the hub to the interior face of the skirt. Each respective spoke (23), (25) has respective concave faces (26) and (27) that face the ball (2) in the housing which has a similar radius of curvature to that of the ball, and is of T-shaped cross section having a strengthening base flanges (28) from which a tapering wall (29) extends upwardly. Each free spoke has at it free end a pimple (30) proud of the concave face (27) which spaces that face (27) from the ball (2) and the pimples (30), being symmetrically arranged, centre the ball. The spokes (23, 25) act as wiper blades regulating the depth of film adhering to the surface of the ball (2) as the ball rotates.

The bottle (1) comprises a head section (36) of circular cross section having an axis inclined at an angle of about 22° to a front wall (37) of a body (38) of oval transverse cross section, which front wall is almost perpendicular to flat base (39) that permits the dispenser to stand upright. To its rearward side, head (35) has a more sharply inclined base wall (40) that extends approximately to beneath the axis of the head (36) and intersects with a sloping rear wall of the body (38) which is approximately aligned with the head axis. The head base wall (40) forms with body rear side-wall (41) a shelf to support the index finger of the hand that grasps the dispenser in a handshake, rear side-wall (41) being pressed into the palm of the hand.

The cap (5) has a top-wall (31) having a flat outer surface enabling the dispenser to be stood in an invert orientation and a centrally located annular dependent wall (32) which can contact the ball (2) to urge it against the sealing ring (17) and a side-wall (33) having on its interior face an inward-facing annular shoulder (34) which can contact the upper wall (14 of the housing (3) and urge it against the ball (2) and on its exterior face a screw thread (35) for co-operation with a corresponding thread (16) on the bottle head (36). When the cap (5) is being fitted to the bottle (1) over housing (3), the cap rotation around the housing is converted by the co-operating screw threads (16, 35) into relative axial movement, so that the ball (2) is urged by annular wall (32) towards the bottle (1) and likewise shoulder (34) acting against upper wall (15) of the housing (3) in turn acts on ball (2). In turn, the ball (2) being in contact with the pimples (30) on the free spokes (25) flexes the spider (4) and in particular the free spokes (25) towards the interior of the bottle (1). When the cap is removed, the spider (4) and in particular the free spokes (25) return to their rest positions due to their resilient character. In that rest position, the concave face (26, 27) of the spokes (23) and (25) create with the ball outer surface an annular gap (36) of reasonably constant depth defined by the height of the pimples (30). When the dispenser is in its upright orientation and as the ball (2) rotates) it encounters the leading edges of the spokes (23) and (25) and excess liquid is wiped away leaving a film of desired thickness on the ball, the remainder falling back into the bottle (1).

1. An antiperspirant or deodorant composition in the form of an oil-in-water emulsion comprising:
a continuous aqueous phasein which is dissolved or dispersed an antiperspirant or deodorant active;
a dispersed oil phase;
a nonionic emulsifier or mixture of emulsifiers and
a dispersed particulate fumed silica in an amount of not greater than 2% by weight of the composition.

2. A composition according to claim 1 in which the emulsifier or mixture comprises an alkoxylated aliphatic alcohol.

3. A composition according to claim 2 in which the emulsifier or mixture comprises a ceteth, steareth or ceteareth emulsifier.

4. A composition according to claim 1 in which the emulsifier or mixture has an average degree of ethoxylation of from 5 to 12.

5. A composition according to claim 4 in which the emulsifier or mixture has an average degree of ethoxylation of from 7 to 9.

6. A composition according to claim 1 in which the emulsifier or mixture of emulsifiers has a mean HLB value of from 5 to 12.

7. A composition according to claim 6 in which the emulsifier or mixture of emulsifiers has a mean HLB value of from 7 to 9.

8. A composition according to claim 3 in which the mixture of emulsifiers comprises a mixture of a lower HLB emulsifier having a degree of ethoxylation of from 2 or 3 and a higher HLB emulsifier having a degree of ethoxylation of from 10 to 40.

9. A composition according to claim 8 in which the first and second emulsifiers are present in a weight ratio of from 2:1 to 5:1.

10. A composition according to claim 1 in which the proportion of the emulsifier is from 2 to 4% by weight.

11. A composition according to claim 1 in which the oil phase comprises a plant oil.

12. A composition according to claim 10 in which the plant oil is a triglyceride oil.

13. A composition according to claim 1 containing from 1 to 5% by weight of the oil.

14. A composition according to claim 12 containing from 1.5 to 4% by weight of the oil.

15. A composition according to claim 1 in which the silica is a fumed silica.

16. A composition according to claim 15 in which the fumed silica comprises a hydrophobic silica.

17. A composition according to claim 1 which contains from 0.1% by weight of the silica.

18. A composition according to claim 17 which contains from 0.3 to 1.5% by weight of the silica.

19. A composition according to claim 18 containing from 0.5 to 1.0% by weight of the silica.

20. A composition according to claim 1 in which the silica has an average particle size of up to 500 nm.

21. A composition according to claim 1 in which the silica i present in a weight ratio to water of from 1:275 to 1:75.

22. A composition according to claim 1 which contains from 0.3 to 1.5% by weight of a fragrance.

23. A composition according to claim 1 in which the total weight proportion of emulsifiers and oils is not greater than 7.5%.

24. A composition according to claim 23 in which said weight proportion is from 5.5 to 7.0%.

25. A composition according to claim 23 which contains at least one higher HLB emulsifier in a weight proportion of from 0.5 to 1.0%.

26. A composition according to claim 1 having a viscosity of from 1800 to 5500 mPA·s at 25° C.

27. A process for making an antiperspirant or deodorant oil-in-water emulsion comprising the step of mixing together ingredients comprising water, an antiperspirant or deodorant active, an oil-in-water emulsifier or mixture of emulsifiers, and an oil and shearing the mixture to disperse the oil as droplets within the water characterised in that the ingredients are in accordance with claim 1.

28. A process according to claim 27 in which the mixture of ingredients is obtained by preparing an aqueous phase and an oil phase separately and mixing the two phases prior to shearing the mixture of phases.

29. A process according to claim 28 employing the mixture of emulsifiers in which one having an HLB value of <6.5 is incorporated with the oil phase and a second having an HLB of >6.5 is incorporated with the aqueous phase.

30. A process according to claim 28 in which the silica is incorporated with the aqueous phase.

31. A method of improving the drying characteristics of an oil in water emulsion containing an antiperspirant or deodorant which comprises incorporating into the emulsion not greater than 2% by weight of a particulate silica forming a composition in accordance with claim 1.
